(19) United States
(12) Patent Application Publication
(10) Pub. No.: US 2013/0085461 A1
(43) Pub. Date: Apr. 4, 2013

(54) Method for Pre-Debriding Treatment of Non-Viable Skin Tissue and Compositions and System Thereof

(71) Applicants: BIOMEDICAL CONCEPTS LLC, New River, AZ (US); DERMAL THERAPY (BARBADOS) INC., Christ Church (BB)

(72) Inventors: Donald H. LEAMAN, New River, AZ (US); Alan Lorne PERLMUTTER, London (CA)

(73) Assignees: DERMAL THERAPY (BARBADOS) INC., Christ Church (BB); BIOMEDICAL CONCEPTS LLC, New River, AZ (US)

(21) Appl. No.: 13/645,150

(22) Filed: Oct. 4, 2012

Related U.S. Application Data
(60) Provisional application No. 61/543,148, filed on Oct. 4, 2011.

(51) Int. Cl. B65D 25/04; A61K 31/7004; A61K 38/46; A61M 37/00; A61P 17/00; A61P 31/04; A61P 29/00; A61K 31/17; A61P 17/02

(52) U.S. Cl. 604/291; 514/588; 514/23; 424/94.66; 220/500

(57) ABSTRACT

A pre-debriding composition comprises a chaotropic agent, preferably urea, and, preferably, exfoliating and analgesic agents. The pre-debriding composition reduces the collagen denaturation temperature, Tm, at the site of a wound. Use of the composition along with a supply of heat increases the effectiveness of a subsequent enzymatic debriding treatment. A dressing and heating system is also provided for use with the pre-debriding composition. An “instant activated” debriding composition and package is provided comprising a gel composition and a powdered debriding zymogen composition, wherein mixing of the two compositions results in an activated enzymatic debriding composition. 22290368.1

### CROSS REFERENCE TO RELATED APPLICATIONS

The present application claims priority under the Paris Convention to U.S. Application No. 61/543,148, filed Oct. 4, 2011, the entire contents of which are incorporated herein by reference.

### FIELD OF THE INVENTION

The present invention relates to methods for improving the efficiency of enzymatic debridement of necrotic tissue. More particularly, the invention relates to methods and compositions for pre-debriding treatment of wound tissue and to methods and systems for such treatment.

### BACKGROUND OF THE INVENTION

Removal of necrotic or non-viable skin tissue which has developed in the wound sites of individuals with chronic pressure ulcers and vascular insufficiency ulcers is a key step in healing such wounds. This process is referred to as debridement. Necrotic tissue prevents normal healing by physically and biochemically obstructing the cascade of healing events in a wound, and commonly harbors pathogenic bacteria organisms and their toxins. Debriding of the necrotic tissue is a first priority in healing such a wound and is commonly accomplished by: 1) surgical debridement; 2) mechanical debridement; 3) autolytic debridement; or 4) enzymatic debridement. Surgical debridement and mechanical debridement, while effective, are often somewhat non-specific and can result in excessive tissue removal and pain to the patient. Autolytic debridement is used in cases where the amount of necrotic tissue is relatively small and the slow rate of self-debridement is acceptable. Enzymatic debridement, using pepsin or papain/urea, is more rapid and highly selective for non-viable tissue only. Examples of known enzymatic debridement methods and compositions include those taught in U.S. Pat. No. 6,548,556 and US Patent Application published under number 2009/0010910, as well as commercially known compositions such as Accuzyme®.

Although enzymatic debridement methods are common, it has been found that the condition of the Type I collagen in the dermis and epidermis strongly affects the length of time commercially available pepsin or papain/urea ointment takes to digest the collagen. Necrotic tissue in wounds caused by burns, either chemical or thermal, are quite rapidly digested by pepsin or papain/urea. By comparison, necrotic tissue in wounds which are caused by pressure or vascular insufficiency are slowly digested, taking approximately twice as long. The difference is that the collagen triple helix in the burn wound necrotic tissue has been denatured and remains in that condition following the chemical or thermal insult. The necrotic tissue in the pressure ulcer or vascular insufficiency ulcer is the result of a lack of blood supplied oxygen and remains in its folded or “native” orientation and is, therefore, much more resistant to digestion by pepsin or papain.

Native form Type I collagen can be denatured by thermal exposure at a specific elevated temperature for a specific time. The denaturation temperature, Tₘ, is related to the amount of water surrounding the collagen triple helix. For example, as noted by Miles et al. [3], at more than 6 moles water per tripeptide, the enthalpy of denaturation on a dry tendon basis was found to be independent of hydration at 58.55±0.59 Jg⁻¹. Between about 6 and 1 moles water per tripeptide, dehydration caused a substantial loss of enthalpy of denaturation, caused by loss of water bridges from the hydration network surrounding the triple helix. At very low hydrations (less than 1 mole of water per tripeptide), where there was not enough water to form bridges and only sufficient to hydrogen bond to primary binding sites on the peptide chains, the enthalpy was approximately constant at 11.6±0.69 Jg⁻¹. The authors found that this was assigned mainly to the breaking of the direct hydrogen bonds between the alpha chains.

While the denaturation temperature (Tₘ) of human Type I collagen isolated from the body has been determined to be near body temperature (36-40° C.) by various investigators, “aging” of Type I collagen in the body was found to raise the shrinkage temperature of reconstituted fibrinous gels by 4° to 6° C. and greatly increase resistance to dissolution at high temperatures. (Gross, J. [4]). The newly formed fibrils were found to dissolve without shrinking, whereas older gels exhibited shrinkage before dissolution. Furthermore, the rate of heating collagen to achieve denaturation has pronounced effect on that temperature. Gradually raising the temperature to denaturation temperature results in a lower Tₘ temperature than rapidly increasing the temperature, especially where the collagen concentration is high. (Matusushita

Scientific research in the biophysics of collagen has shown that certain chemical agents can reduce the denaturation temperature, Tₘ, by reducing the water associated with the collagen triple helix. In the science of leather making, recent work on a mammalian model (Usha et. al. [1]) found that 3M urea and 10 mole % n-propanol can be demonstrated by differential scanning calorimetry and viscosity measurements to reduce the denaturation temperature of non-viable rat tail Type I collagen (Tₘ=67.5° C.) by 13.5° C. and 3.5° C., respectively.

Debridement of soft tissue necroses with much higher levels of urea (40%) has been shown to be effective in patients with amputated legs. Such chemical debridement, without additional heat, was accomplished over 72 hours using a 40% urea paste that was carefully controlled to contact only the subject necrotic tissue by applying a ½ inch frame of liquid adhesive, cloth tape, more liquid adhesive, polyethylene film, and two more layers of cloth tape. (Pelle et al. [6]). As would be known to persons skilled in the art, the reason for such precaution is related to the damaging effects on skin caused by high concentrations of urea.

Research by others has shown that the introduction of glucose and sodium lauryl sulfate at defined levels aided in maintaining the denatured condition of the collagen, i.e. these compounds support the transition from the collagen triple helix into random coils (gelatin).

It has long been known in the art of skin resurfacing that certain unsaturated fatty acids, i.e. alpha hydroxy acids, are effective in removing non-viable epithelial tissue. Lactic, malic and mandelic acid were selected for their effectiveness and stability for incorporation in this composition to remove such non-viable epithelial tissue in the wound and wound margins and encourage post-debridement re-epithelialization from the wound edges.

There exists a need for improving the efficacy of enzymatic debridement methods.

### SUMMARY OF THE INVENTION

In one aspect, the present invention provides a method wherein a pre-debriding composition is applied to a wound, along with heat, in order to denature collagen at a wound site that is to be subjected to enzymatic debridement. Such pre-debriding treatment has been found by the inventors to improve the efficiency of the enzymatic debridement treatment.

Thus, in one aspect, the present invention provides a pre-debriding composition comprising a first chaotropic component. In one aspect, the first chaotropic component is urea. In a further aspect, the composition comprises at least one other, second, chaotropic agent, such as an alcohol. In a preferred embodiment, the composition comprises at least one analgesic component.

In another aspect, the invention provides a method of treating a wound site prior to debridement, the method comprising:
a) applying to the wound site, at least one first chaotropic agent and at least one analgesic agent, for reducing the collagen denaturation temperature at the wound site; and,
b) applying heat to the wound site following application of the pre-debriding composition, said heat being applied for a period of time to cause denaturation of collagen at the wound site.

In another aspect, the invention provides a system for heating a wound site following application of a pre-debriding composition to cause denaturation of collagen at the wound site, the system comprising:

i) a first dressing layer for placement over the wound site, the first dressing layer comprising a non-absorbent material;

ii) a temperature measuring or sensing means for positioning over the first dressing layer;

iii) a second dressing layer, for placement over the temperature measuring or sensing means and over the first dressing layer, the second dressing layer comprising an absorbent material having a non-adherent layer, the second dressing layer including a fenestration corresponding to the wound site being treated; and,

iv) a heating means for heating the wound site.

In another aspect, the invention provides a kit for providing a pre-debriding treatment at a wound site comprising:

a) a container containing a pre-debriding composition according to the invention;

b) a first dressing layer comprising a non-absorbent material;

c) a temperature measuring or sensing means;

d) a second dressing layer comprising an absorbent material having a non-adherent layer;

e) a heating means for heating the wound site; and, optionally,

f) instructions for using components (a) to (e).

In another aspect, the invention provides a composition for enzymatic debriding with pepsin, wherein the composition is provided in two components consisting of a gel composition and a powder composition, wherein the powder composition comprises pepsinogen and wherein the gel composition is adapted to activate pepsinogen to form pepsin when the two components are mixed.

In another aspect, the invention provides a package for the debriding composition of the invention, wherein the package comprises two compartments separated by a breakable divider, one of the compartments containing the gel composition and the other of the compartments containing the powder composition.

### BRIEF DESCRIPTION OF THE DRAWINGS

The features of the invention will become more apparent in the following detailed description in which reference is made to the appended drawings wherein:

FIG. 1 is cross sectional schematic view of a system according to an aspect of the invention for applying heat to a region being treated with the pre-debriding composition of the invention.

### DETAILED DESCRIPTION OF THE INVENTION

The terms “comprise”, “comprises”, “comprised” or “comprising” may be used in the present description. As used herein (including the specification and/or the claims), these terms are to be interpreted as specifying the presence of the stated features, integers, steps or components, but not as precluding the presence of one or more other feature, integer, step, component or a group thereof as would be apparent to persons having ordinary skill in the relevant art.

The present invention provides, in one aspect, a pre-debriding wound treatment composition that is applied to the treatment site prior to an enzymatic debridement treatment, to enhance the effectiveness of the latter. The pre-debriding composition comprises a skin tissue (collagen) denaturant, and preferably an exfoliant and/or a topical analgesic, for application to ulcerations and other such wounds containing non-viable skin tissue. The composition may be provided, for example, in the form of an ointment or a gel, although other administration forms are possible. The pre-debriding composition of the invention significantly increases the effectiveness of subsequently applied enzymatic compositions.

In one aspect, the enzymatic compositions comprise one or more proteolytic enzymes. Preferably, the proteolytic enzymes comprise one or more of pepsin, fruit proteolytic enzymes, such as papain, actinidin, bromelin, ficin, or bacterially derived proteolytic enzymes. More preferably the enzymes are pepsin or papain. The fruit proteolytic enzymes may be delivered in crude or purified form to the necrotic tissue of the wound. Such fruit enzymes may be provided in a variety of preparations such as, but not limited to, gels, pastes, ointments, salves, or foams. Preparations containing fruit enzymes may preferably contain fructose and/or glucose and more preferably may contain antimicrobial agents such as methylglyoxal or its precursor 1,3-dihydroxyacetone. In one embodiment, the one or more proteolytic enzymes are provided in their zymogen form.

As used herein, the term “zymogen” will be understood to mean an inactive form of an enzyme that is converted to its active form by action of an activating agent. Such activating agent may include an acid, another enzyme or a combination thereof.

The enzymatic compositions may also include urea.

The pre-debriding composition significantly reduces the time to attain enzymatic debridement. The pre-debriding composition of the invention is not limited to use with any particular enzymatic debriding formulations.

In another aspect, the invention provides a system that is applied after application of the pre-debriding composition and which serves to raise the temperature of the treatment area to a desired temperature. After application of the composition, that is preferably in the form of an ointment or a gel, the system according to an aspect of the invention is used to dress the wound and warm the skin under the wound dressing to preferably about 10° C. above normal body temperature, such as, for example, 47°±2° C. Such system or dressing may be provided for a period of 2-6 hours following application. The system of the invention is described further below. As will be understood, the system of the invention provides a highly localized and controllable heating of the treatment region.

In one aspect, the present invention provides a method for treating a wound site prior to pre-debridement, which comprises application of a pre-debriding composition, as discussed above, and the application of heat to the treatment site. The method results in reduced exposure time to enzymatic debridement formulations without exposing the patient to potentially caustic levels of chemicals (such as urea). The system of the invention allows the required heat to be applied locally to the wound site to facilitate the activity of the pre-debridement composition and avoids the potential for thermal damage to the patient's skin.

For example, in one aspect, the invention provides a formulation comprising urea at a preferred concentration of 25%, plus several other ingredients for fostering denaturation of Type I collagen. With such formulation, it was discovered by the present inventors that only a 10° C. rise in temperature was needed to denature the necrotic tissue collagen and make it highly susceptible to enzymatic (e.g. pepsin or papain) digestion. Although pepsin and papain are exemplified herein, other enzymes or enzyme combinations may be used. Further, by raising the temperature slowly over 30-60 minutes, an increase in the denaturation temperature (Tₘ) was avoided. Such temperature increase is efficiently achieved using a system according to another aspect of the invention as described herein. It should be noted that, although urea was common in papain-containing debriding ointments (which were removed from the United States market January 21, 2009), none of such known formulations exceed a urea concentration of 10%, and none are used above the wound temperature of 30-37° C. In fact, warming urea based formulations would be counterintuitive due to the possibility of releasing ammonia.

Without being bound to any specific theory, the pre-debriding composition of the present invention, containing levels of the ingredients described herein, significantly reduces the temperature of denaturation and contributes to the transition from collagen triple helix to random coils. The combination of such a composition with the system of the invention described herein, which raises the skin/wound temperature slowly and then holds it elevated approximately 10° C. above body temperature, results in the denaturation of Type I collagen in non-viable tissue without concern for thermal injury to the patient. As will be understood, viable, vascularized wound tissue surrounding and underlying the necrotic tissue is capable of dissipating the thermal energy applied to the wound site, while the non-viable tissue cannot, and therefore it is “targeted” for warming to the full temperature increase caused by the warming device, as moderated by the wound dressing. Furthermore, the application of the pre-debriding composition (e.g. ointment) on the necrotic tissue forms a thermal conductive coupling from such tissue, through a dressing forming part of the system, to a warming or heating device of the system, which will also concentrate the thermal energy on the non-viable tissue. Additionally, according to a preferred embodiment of the invention, the intact skin surrounding the wound site is protected from full thermal exposure by cutting a wound shaped opening in one wound dressing component of the system.

A) Pre-Debriding Composition of the Invention

According to one aspect, the present invention comprises a composition for application to a wound site prior to an enzymatic debriding treatment. As discussed above, the pre-debriding composition of the invention serves primarily to denature collagen in order to reduce the time required for the following enzymatic debriding step. For this purpose, the composition of the invention contains one or more chaotropic agents, or components, and preferably one or more analgesic agents or components, and more preferably unsaturated fatty acids and one or more agents or components for exfoliation. The composition of the invention is preferably in the form of an ointment or a gel that is capable of being topically applied to a wound area. As such, the composition will be understood to include one or more carriers, diluents, excipients, emulsifiers, thickening agents and/or preservatives etc. as would be known to persons skilled in the art for formulating such ointments or gels.

In one aspect, the composition serves to soften, denature, exfoliate and provide analgesic pain relief to the area of the skin to which the composition is applied. The analgesic component is preferred so as to reduce any pain or discomfort of the skin associated caused by the contact with the chaotropic agents of the composition, which would otherwise cause a stinging sensation due to their dehydrating effect on nerve endings. The composition of the invention is applied onto skin defects or wounds containing non-viable tissue. Such non-viable skin tissue may be partial or full thickness, necrotic by virtue of vascular insufficiency or prolonged pressure on the skin, or may have become eschar, i.e. hardened necrotic tissue such as can result from surgical amputation, excision, or traumatic injury, where substantial skin tissue has “died back and dried out.” The function of the composition of the invention is to prepare the necrotic tissue for digestion and removal by an enzymatic debriding agent, such as a pepsin or papain based agent, which would be subsequently applied. The composition of the invention results in the denaturation of the Type I collagen in non-viable skin tissue, which allows the pepsin or papain to readily and much more rapidly digest such tissue. In a preferred aspect, the composition of the invention further exfoliates such tissue around the edges of the wound to remove squamous epithelium so as to provide a “fresh” wound edge for healing.

Agents that break up dense macromolecular and lipid-rich domains are known as chaotropic agents. In cosmetology, their activity is the basis for improved hydration and moisturization of otherwise rough, scaly and dry skin. Anchordoguy et al. [8] showed that alcohols, amides and urea act as chaotropic agents. Urea, substituted ureas, amides and dimethyl sulfoxide (DMSO) are known chaotropic agents, which may be used in the composition of the present invention. Urea itself shows some lipid solubility as well. Of particular importance to this invention are the abilities of this class of compounds, particularly urea, to diffuse rapidly in aqueous solutions and to break up dense macromolecular domains of fibrous and globular proteins. Under the influence of 25% urea and preferably other chaotropic agents in the formulation, Type I collagen molecular bonds that normally unite the triple helix are reduced to only weak, non-covalent bonds (hydrogen, polar, and hydrophobic contacts, etc.) The collagen weakening effect of the composition is a major causative factor in the denaturation of necrotic tissue Type I collagen, particularly when used in conjunction with applied heat energy.

A pharmaceutically acceptable chaotropic agent of the type discussed above also plays an important role in facilitating penetration of the analgesic agent into the skin, where such agent is present in the composition. In one embodiment, the composition of the present invention comprises from about 15 to about 40 wt %, and preferably about 20 to about 30 wt %, of a chaotropic agent. In one aspect, the chaotropic agent may comprise: urea; N-alkyl-substituted urea or N,N-dialkyl-substituted urea having from 6 to 9 C-atoms (i.e. C₆-C₉) in the alkyl group; amides of formula (R²)₂N—CO—R¹—R³, wherein R¹ represents a chemical bond or an alkyl group having from 1 to 14 C-atoms and R² and R³ are independently hydrogen atoms or methyl groups; dimethyl sulfoxide (DMSO); or combinations thereof. In a particularly preferred embodiment, the chaotropic agent used is urea, provided at a concentration of 20-30 wt %, and more preferably at a concentration of 25 wt %.

In addition to being mild analgesic agents themselves, pharmaceutically acceptable alcohols have been shown to possess chaotropic properties and to increase penetration of topically applied agents. Penetration and chaotropic activity improve with increased length of the hydrophobic hydrocarbon chain. See, for example, Behl et al. [9]. According to a preferred embodiment of the invention, the composition may comprise or further comprise a chaotropic agent comprising an alcohol. Such alcohol is preferably selected from aliphatic alcohols having from 2 to 8 C-atoms in the alkyl chain, and aromatic alcohols such as benzyl alcohol, or combinations thereof. In a preferred embodiment, the composition comprises from about 3 to about 5 wt % of such alcohol, and more preferably 4 wt %. In one preferred embodiment, the composition comprises n-propanol at a concentration of about 3-5 wt %, and preferably 4 wt %.

Penetration of the composition of the invention can be further enhanced by the addition of unsaturated long chain fatty acids. Long chain unsaturated fatty acids act to open channels in the epidermal cell barrier by increasing hydration, moisturization and break-up of lipid-rich domains of the skin surface. The fatty acids of the invention may comprise a-hydoxy acids. Therefore, the composition of the invention may optionally comprise an unsaturated fatty acid such as: oleic acid, linoleic acid, linolenic acid, lactic acid, malic acid, mandelic acid, or combinations thereof. In a preferred embodiment, the unsaturated fatty acid used is oleic acid. In a preferred embodiment, the fatty acid is present in the composition in concentration of about 1 to about 5 wt %.

In a preferred embodiment, the pre-debriding composition of the invention further comprises an agent to prevent or inhibit re-naturing of the triple helix of the Type I collagen. Such agents, or re-naturing inhibitors, will be known to persons skilled in the art and may comprise glucose, related glucose sub-unit products, protein solubilization and emulsification agents (i.e. detergents), alcohols or combinations thereof. In one embodiment, the protein solubilization and emulsification agent is sodium lauryl sulfate. In one embodiment, the alcohol may be n-propanol or other such alcohols. In a preferred embodiment, glucose is used as the re-naturing inhibitor. The concentrations of the re-naturing inhibitor would be known to persons skilled in the art. For example, and without limiting the scope of the invention, the following concentrations may be used: glucose: 1-5 wt % (preferably 2 wt %); n-propanol: 2-10 wt % (preferably 4 wt %); and sodium lauryl sulfate: 0.005-0.015 wt % (preferably 0.01 wt %).

As indicated above, the composition of the invention may include an analgesic agent. The analgesic agent is responsible for analgesia at the site of application, primarily in order to minimize or eliminate the stinging sensation associated with the application of chaotropic agents such as urea. A number of topical analgesic agents as known in the art may be used in the pre-debriding composition of the present invention, such as benzocaine, butamben picrate, dibucaine, dimethisoquin, diclonyne, lidocaine, pramoxine, tetracine, and pharmaceutically acceptable salts thereof. In the composition of the present invention, the analgesic agent typically comprises from about 0.2 to about 20 wt % of the final composition. According to a preferred embodiment, the analgesic agent used is benzocaine, lidocaine, or a pharmaceutically acceptable salt thereof. In a particular embodiment, the analgesic agent comprises lidocaine hydrochloride, at a concentration of about 4-5 wt %, which is known to be effective when applied to viable tissue at relatively low concentrations. Lidocaine is further preferred since it serves to reduce the pH of the composition.

A pharmaceutically acceptable emulsifier may also be provided in the composition to improve the availability of the analgesic agent, which may have a limited miscibility with lipids. The emulsifier can further enhance the penetration by facilitating the emulsification with hydrophobic substances that make up the natural barrier of the skin—lipids, cholesterol and ceramides. Various emulsifiers known in the art may be used in the composition of the invention. For example, in addition to being a suitable emulsifier, phosphatidyl cholines, or lecithins, interact with alcohols and enhance their analgesic action. Using pure model systems, Rowe [10] showed that ethanol disruption of lipid structures increases when the number of C-atoms in saturated acyl side chains of di-substituted 1,2-glycero-3-phosphatidyl choline increases from 14 to 21. Naturally occurring lecithins may be derived from a variety of sources (e.g. soybeans, egg yolk etc.), and generally represent mixtures in which substituents comprise various percentages of acids. In one example, soybean lecithin is defined as comprising 11.7% palmitic acid, 4.0% stearic acid, 8.6% palmitoleic acid, 9.8% oleic acid, 55.0% linoleic acid, 4.0% linolenic acid and 5.5% of C₂₀-C₂₂ acids. Encompassed within the scope of the present invention are both synthetic phosphatidyl cholines (e.g. products supplied by Avanti, Birmingham, Ala.), and naturally occurring lecithins (e.g. products supplied by American Lecithin, Danbury, Conn.). In one embodiment of the present invention, therefore, the composition comprises lecithin, selected from: di-substituted 1,2-glycero-3-phosphatidyl cholines with 14 to 24 C-atoms in saturated acyl side chains thereof; purified natural lecithins, such as soybean or egg yolk lecithin; and mixtures thereof. In a preferred embodiment the emulsifier may be present in the composition in a concentration from about 1 to about 5 wt %.

In a preferred embodiment, the composition of the invention is prepared in the form of a stable gel or ointment. Pharmaceutically acceptable gelling and thickening agents that can be used are carboxypolymethylene (a vinyl polymer with active carboxyl groups, e.g. Carbopol™940 supplied by B. F. Goodrich), xanthan gum (e.g. a product supplied by Rhone-Poulenc, Inc. under the name of Rhodigel™), hydroxymethylpropyl cellulose (e.g. a product supplied by the Dow Chemical Company under the name of Methocel™), magnesium aluminum silicate (such as Veegum™, a hydrated magnesium aluminum silicate supplied by R. T. Vanderbilt, Inc.), polyethylene glycol (such as PEG-400™ supplied by the Dow Chemical Company), and combinations thereof. Various other gelling and thickening agents will be known to persons skilled in the art. According to one embodiment of the invention, the composition is in the form of a gel, wherein the gelling agents used are Carbopol™ 940 and PEG-400™. The gel will preferably have a viscosity within the range of about 3 to about 6 Cps (Centipoises). To ensure stability of the gel, the pH of the composition is adjusted to about 5 by the addition of a pharmaceutically acceptable organic base, for example triethanolamine. The gelling and/or thickening agent is preferably present in an amount of about 0.1 to about 10 wt %.

In order to maintain desired properties over prolonged periods of time, the composition can be supplemented with one or more pharmaceutically acceptable preservatives as known in the art. For example, the preservatives that can be used in the present invention include, for example, methyl paraben, propyl paraben, imidazolidinyl urea, tetradecyl-trimethyl ammonium bromide (Cetrimide™), sodium benzoate, thymol, and combinations thereof. Various other preservatives will be known to persons skilled in the art.

B) Method of the Invention

As discussed above, in one aspect, the invention provides a pre-debridement method wherein a pre-debriding composition is applied to a wound or other such treatment site or area, along with heat. Preferably, the treatment area is heated by about 10° C. Such method has been found by the inventors to accelerate the subsequent enzymatic debridement of the treatment site. In another aspect, the method of the invention comprises the application of heat and the pre-debriding composition for a period of time, followed by application of an enzymatic debriding formulation.

As mentioned above, the pre-debriding composition of the present invention has been found by the inventors to reduce the temperature of denaturation of collagen of the epidermal and dermal layers of the skin. As a result, an enzymatic debridement of the treatment area is accelerated since the skin in such area is rendered more susceptible to enzymatic digestion by agents such as pepsin or papain. In this manner, the method of the invention allows for selective debridement of the treatment region.

C) System of the Invention

According to one aspect of the invention, a system is provided for use in conjunction with the pre-debriding composition described above. As will be understood, the system of the invention serves to facilitate implementation of the aforementioned method. In general, the system of the invention serves to heat the treatment site, where debriding is needed. The combination of such application of heat along with the pre-debriding composition serves to increase the efficiency of a subsequent enzymatic debriding treatment. It will be understood that any device that supplies the required heat to the treatment site would be sufficient. However, in one aspect, the invention provides a unique heating system that safely provides the required heat in a highly localized manner.

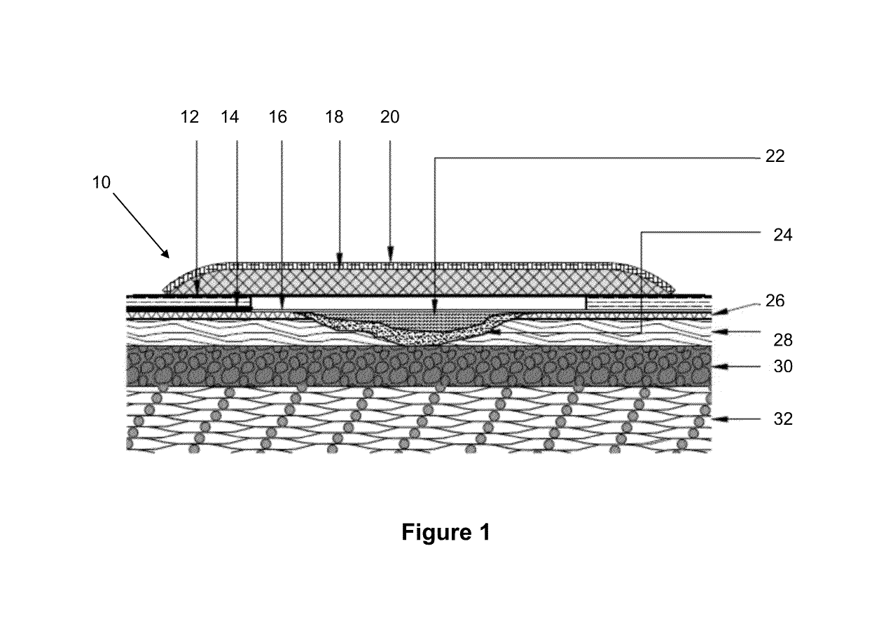

An example of a system according to a preferred embodiment of the invention is illustrated in FIG. 1. As shown, a system 10 according to an aspect of the invention comprises several components, which are preferably arranged in a layered structure. As shown, the system 10 of the invention preferably comprises:

i) An absorbent wound dressing 12, such as a Telfa™ dressing (Kendall Co.), or similar absorptive dressing, preferably having a non-adherent layer to face the skin being treated;

ii) A temperature measuring or sensing means 14, such as a liquid crystal thermometer (e.g. a thermometer supplied by American Thermal Instruments);

iii) A non-absorbent, preferably transparent wound dressing 16;

iv) A heating means 18 for heating the area being treated. In one embodiment, the heating means may comprise an atmospheric-oxygen activated warming device containing iron powder, water, activated charcoal and vermiculite in a fabric pouch and sealed in a foil wrap, such as that by HeatMax, Inc., Dalton, Ga., or equivalent;

v) Self-adherent wrap 20 to secure the heating means and dressings in place.

The system 10 of the invention serves to provide a gradual warming of the wound site on which the composition 22 has been applied. For example, the heating means 18 of the system 10 may be adapted to provide the necessary heating over a time period such as 30-60 minutes. The heating means 18 may also be adapted to maintain a desired temperature for several hours, such as 2-6 hours, and preferably 4 hours. The system 10 provides a sterile dressing material for direct contact on the wound site, which protects the wound site from infection. Further, the dressings diffuse the heat energy applied so to attain an even and safe temperature on the skin and wound site. The self-adherent wrap 20 provides a means to secure the system 10 in position above the wound site.

It will be understood that the aforementioned elements of the system are provided to illustrate preferred embodiments of the invention. It will be understood that the main purpose of the system of the invention is to provide a heating means (such as that shown at 18 in FIG. 1). The other elements of the system as mentioned above assist in the placement of the heating means 18 at a desired location or position and to provide other beneficial effects, such as infection control etc.

FIG. 1 also illustrates the epidermis 26, the dermis 28, subcutaneous tissue 30 and muscle tissue 32, adjacent the wound area.

The method of using the system 10 will now be described with reference to FIG. 1. Firstly, an amount of the pre-debridement composition 22, as described above, of the invention is first applied to a wound site. The composition in this instance would preferably be in the form of an ointment or a gel, which is applied onto the necrotic tissue 24 in the wound. Following application of the composition, the system 10 is then applied. In this process, the non-absorbent wound dressing 16 is first placed over the wound site where the composition is applied. The absorbent dressing 12 is then applied over the non-absorbent dressing 16. The region of the wound is traced over the absorbent dressing 12 and the region of the absorbent dressing 12 corresponding to the wound region is cut out and discarded. The remainder of the absorbent dressing 12 is then placed over the wound region, with the non-adherent side facing against the wound. In this manner, the treatment site is bounded by the absorbent dressing 12 but does not cover it. The absorbent dressing 12 is then secured to the skin using medical tape as is commonly known. In this arrangement, it will be understood, that a fenestration or “window” is created in the absorbent dressing 12, wherein the treatment site of the wound is made visible through the transparent, non-absorbent dressing 16. As will be understood, this is a preferred embodiment of the invention and the invention is not limited to having such fenestration.

Next, the temperature measuring means 14 is inserted under the non-absorbent dressing 16. As mentioned above, the temperature sensing means is preferably a liquid crystal thermometer and the remainder of the discussion will refer to such device. It will be understood that various other temperature measuring or sensing means may also be used, in particular those including an electronic or thermocouple containing device and the like. The liquid crystal thermometer is inserted so that the liquid crystal end lies on the transparent, non-absorbent dressing 16, near the cut-opening in the absorbent dressing 12, and with the “handle-end” of the thermometer extending out past the edge of the dressings. The “window area” of the wound site is lightly pressed to ensure adequate contact is made between the necrotic tissue, the composition and the non-absorbent dressing 16. Such contact will function as a thermal coupling to the necrotic tissue when the heating means 18 is applied.

The system 10 of the invention may utilize a variety of heating means 18. However, as discussed above, some commercially available products may be more convenient to use and these will comprise a preferred embodiment of the invention. In the next step, the warming device, i.e. heating means, 18 is removed from its foil package to activate it. The device 18 is then placed on the absorbent dressing 12, taking care not to allow any part of the warmer device to directly contact the skin. For this reason, the absorbent dressing 12 would preferably be larger in size than the heating device 18.

A roll of self-adherent wrap 20 is the supplied to gently, but securely affix the warming device 18 in place. Preferably, when using a wrap 20 in a tape form, no more than three turns is provided over the wound site. The liquid crystal thermometer reads four temperature ranges: 36/37° C., 43/44° C., 48/49° C. and 50/54° C. The last range is marked “TOO HOT”. The instructions will indicate that the 43/44° C. and the 48/49° C. are the lower end and upper end of the ideal temperature range. The lowest range indicates when body temperature is reached (wounds often have temperatures below 37° C., as does skin, which is typically 30-34° C.). The thermometer strip can be quickly removed from between the transparent, non-absorbent dressing 16 and the absorbent dressing 12, read within a couple seconds, and then replaced for later monitoring.

D) Kits of the Invention

As will be understood, the above described system of the invention may be constructed on site to suit the individual patient's needs and using available materials. However, in one aspect of the invention, all of the aforementioned components of the system may be provided in a kit along with the required instructions.

In addition, the kit of the invention may further include a container containing the pre-debriding composition of the invention (such as in an ointment or gel form). The pre-debriding composition may, for example, be provided in a tube or other such container for ease of application and the instructions would be amended accordingly.

As discussed further below, the kit of the invention may also include a debriding formulation, or components of a formulation, for a subsequent debriding step. Such debriding step would preferably comprise enzymatic debriding.

E) Debridinq Composition of the Invention

In another aspect, the invention provides a composition of formulation for conducting a debriding procedure, and more preferably an enzymatic debriding procedure.

Enzymatic debriding agents of the invention may include any proteolytic enzyme or combination of enzymes, such as pepsin or papain (both of which are further described herein), other fruit derived proteolytic enzymes, such as kiwi enzyme (actinidin), pineapple enzyme (bromelin), fig enzyme (ficin), and/or bacterially derived proteolytic enzymes. Fruit proteolytic enzymes may be delivered in crude or purified form to the necrotic tissue of the wound in a variety of preparations. Examples of such preparations include, but are not limited to, gels, pastes, ointments, salves and foams. As discussed further below, preparations containing fruit derived enzymes also preferably comprise fructose and/or glucose and preferably include one or more antimicrobial agents, such as methylglyoxal or 1,3-dihydroxyacetone (which is a precursor of methylglyoxal).

Fruit derived proteolytic enzymes mentioned above, are known in the art and are known to function much like papain (also a fruit proteolytic enzyme) or pepsin on tissue proteins. The function of any of these enzymes is enhanced by the use of the pre-debriding composition and method described above. That is, as described above, the pre-debriding composition and method of the invention serves to “unfold” skin collagen thereby rendering the collagen much more susceptible to proteolytic degradation by the aforementioned enzymes.

Debriding compositions containing the aforementioned fruit derived proteolytic enzymes are preferably in the form of a fructose and/or glucose containing gel. Such formulations also preferably contain at least one antimicrobial agent, such as methylglyoxal, to control microbial contamination. Generally, without such antimicrobial agents, bacteria would rapidly multiply to infectious levels in the wound site, nourished by the collagen degradation products and the fructose and/or glucose contained in the formulation. Methylglyoxal is a preferred antimicrobial agent for the present debriding formulation. As known in the art, methylglyoxal kills a very broad range of bacteria, fungi and many viruses and has been used in other pharmaceutical formulations.

In one embodiment, the invention provides an “instant activated” debriding gel containing an enzymatic debriding composition comprising one or more debriding agents, such as one or more proteolytic enzymes. In one aspect, the debriding composition is formed by adding an activating gel to a dry zymogen powder, that is the zymogen form(s) of the one or more proteolytic enzymes in powder form. Preferably, the zymogen powder also includes a dry lidocaine component.

In one embodiment, the proteolytic enzyme used in the debriding composition is pepsin. As indicated above, the pepsin of the debriding composition is supplied in its pro-form, i.e. its zymogen form, namely pepsinogen. As known in the art, pepsinogen has an additional 44 amino acids, which must be cleaved to provide pepsin. Pepsinogen is activated by acid, which allows it to unfold and cleave itself in an autocatalytic manner, thereby resulting in pepsin (the active form of the enzyme). The function of pepsin is well known in the art and has been found to be very effective in digesting necrotic tissue for its safe removal from wounds. Pepsin functions best in acidic environments and is active in the acidic environment of the “instant activation” gel of the invention, which can be applied to non-viable skin tissue with a pH of 1.5 to 4. Pepsin denatures at a pH greater than 5. Its optimal activated time is measured in hours at skin temperature since it is auto-catalytic and digests itself; hence instant activation is essential for a commercial product. In one aspect, the invention provides such “instant activation” formulation. As will be known to persons skilled in the art, the digesting activity of pepsin is slow even at low pH values and the self-digesting action of pepsin renders it less effective a few hours after application. For this reason, the composition of the present invention would preferably be applied twice daily for efficiently digesting collagen/proteins of necrotic tissue.

One example of a preferred debriding gel formulation of the invention is discussed below in Example 3. As will be understood, the debriding gel is designed to be activated and applied immediately. For this reason, the components of the gel are separated into two parts that can be combined and applied instantaneously. Preferably, the parts comprise 1) a prepared gel formulation and 2) the required active components in dry form (i.e. powder). In a preferred embodiment, the active components comprise pepsin (in its zymogen form) and lidocaine. As will be understood, pepsin serves as an enzymatic debriding agent and lidocaine serves as a topical analgesic agent. It will be understood also that the use of an analgesic agent is a preferred but optional component. Further, the choice of lidocaine as the analgesic agent is also preferred due to its pH reducing effect, which counteracts the acidic nature of the remaining gel components.

In a further aspect, the “instant activated” debriding composition of the invention may be provided in a single package having two separated components. In use, the active components in powder form may simply be combined into the gel component to form the debriding formulation.

In a further aspect of the kit of the invention, the above mentioned packaging containing the debriding formulation components may also be included. It will be understood that the components of the debriding formulation may also be provided in the kit in two separate containers instead of the aforementioned packaging. It will also be understood that where the debriding formulation components are included, the instructions of the kit will be amended accordingly.

### EXAMPLES

Aspects of the invention will now be described with reference to various examples. It will be understood that the following examples are provided to illustrate the present invention and are not intended to limit the scope of the invention in any way.

### Example 1

### Ointment Composition

An example of an ointment composition for use as a pre-debridement treatment according to the present invention was prepared according to the formulation provided below in Table 1.

TABLE 1

| Ingredient | Percent by weight (wt %) |
|---|---|
| Urea | 25.00 |
| Waxes and mineral oils | 10.00 |
| PEG 100 (Carbowax ™) | 6.00 |
| Lidocaine Hydrochloride | 4.00 |
| n-Propanol | 4.00 |
| Lanolin | 4.00 |
| Cetyl alcohol | 3.00 |
| Glucose USP | 2.00 |
| Mandelic acid | 2.00 |
| Malic acid | 2.00 |
| Preservative complex | 0.50 |
| Sodium lauryl sulfate | 0.010 |
| Triethanolamine 99% | adjust pH to 5.0 |
| Sterile deionized water | q.s. to 100.00% |

### Example 2

### Treatment System

A treatment system according to an aspect of the invention, for use with the composition of the invention, was prepared and is described below. The pre-debridement composition was first applied in the form of an ointment as described in Example 1. Following such application, the components of the system of the invention were then applied in the manner described above. The temperature was then measured.

Temperature (° C.) readings were taken by thermocouples on live human skin (upper thigh area) and are listed in Table 2 below. “Wound Site” readings were taken under the transparent, non-absorbent dressing, in the ointment covering the wound site. The “Surrounding Skin” readings were on top of the transparent, non-absorbent dressing, under the Telfa™ dressing (i.e. the absorbent dressing), next to the wound window. The “Warmer” (i.e. heating device) readings were taken on the underside of the Warmer against the Telfa dressing. Note the slow warm-up period and then continuous warmth period out to 6 hours. Room temperature was 25° C. The Warmer was activated at time 0 and immediately placed into position and wrapped with the self-adherent wrap.

TABLE 2

| | | Surrounding Skin | |
|---|---|---|---|
| | | Reading | |
| | Wound Site | “LC Thermometer | |
| Time (minutes) | Reading | Position” | Warmer Reading |
| 2 | 30 | 29 | 35 |
| 5 | 36.2 | 35.9 | 37 |
| 10 | 39.4 | 37.9 | 39.7 |
| 20 | 41.4 | 40.9 | 43.2 |
| 30 | 43.1 | 42.2 | 44.5 |
| 40 | 43.1 | 42.5 | 44.5 |
| 50 | 44.9 | 43.0 | 44.1 |
| 60 | 45.5 | 43.0 | 44 |
| 75 | 45.4 | 43.0 | 43.9 |
| 90 | 46.4 | 43.1 | 43.4 |
| 105 | 46.4 | 43.0 | 43.4 |
| 120 | 46.4 | 43.6 | 43.2 |
| 150 | 45.1 | 42.8 | 43.4 |
| (air draft stopped) | | | |
| 180 | 46.4 | 43.9 | 43.1 |
| 210 | 45.9 | 44.4 | 43.6 |
| 240 | 47.3 | 45.2 | 44.2 |
| (blanket added) | | | |
| 270 | 47.1 | 45.9 | 44.2 |
| 300 | 47.1 | 45.8 | 44.6 |
| 330 | 47.4 | 47.9 | 45.2 |
| 360 | 47.2 | 47.9 | 45.0 |

In the above described system, the following several factors may influence the actual temperature in the wound site: 1) room temperature; 2) air drafts on wound site; 3) patient's body temperature; 4) patient's skin temperature; 5) wound area/depth; 6) patient's activity level; 7) technique of dressing application; and 8) elevated oxygen levels in room air.

Note that the wound site temperature is about 2-3° C. higher than the Surrounding Skin temperature where the LC (liquid crystal) thermometer is placed in practice. The desired condition for effective denaturation of non-viable tissue saturated with the ointment of Example 1 is 45-49° C. for 2-4 hours. Considering the 2-3° C. offset, if the LC Thermometer indicates that the temperature is not reaching the lower end of the desired range, i.e. 43/44° C., between 1-3 hours elapsed time, measures should be taken to maintain the proper temperature. For instance, stop an air draft or add a light blanket on top of the system and monitor the temperature to achieve the desired level. See above at time points 150 minutes and 240 minutes, for example, where this was done. Conversely, if the LC thermometer temperature starts to get too high, remove the covering blanket or use a fan to reduce the air temperature. While 4 hours is an optimum time for “normal denaturation”, older patients, or patients with severely aged skin will benefit from longer treatment, such as up to 6 hours.

### Example 3

### Instant Activated Pepsin-Lidocaine Debriding Gel

An “instant activated” debriding gel according to an aspect of the invention was prepared, the details of which are provided below in Table 3. As shown, the formulation was prepared in two parts, comprising the gel portion and a dry, powder portion.

TABLE 3

wt %

Part #1: Gel

| | |
|---|---|
| Phase A | |
| Deionized water | 87.9 |
| Xanthan gum | 0.6 |
| Glycerin | 4 |
| Allantoin | 0.3 |
| Disodium EDTA | 0.1 |
| Mandelic acid | 2 |
| Phase B | |
| Phenoxyethanol | 1 |
| Subtotal | 95.9 |

Part # 2: Dry Powder

| | |
|---|---|
| Lidocaine HCL | 4 |
| Pepsin (210 IU or 420 IU) | 0.1 |
| Subtotal | 4.1 |
| TOTAL | 100 |

The above formulation was made immediately before application to non-viable skin tissue which was denatured by the combined use of the composition of Example 1 and the system of Example 2 for a minimum of 2 hours, and preferably 4 hours. The formulation was made by combining and gently mixing 6.713 g of the gel Part# 1 with 0.287 g of the powder of Part #2. This mixing may be accomplished in a special breakable-septum device containing pre-measured amounts of Part #1 and Part #2 may be available to the user for commercial application. Such device, or package, may be included in the kit of the invention as described above. The pH of the gel was 2.25.

Once the gel is mixed with the pepsin powder, the pH of the mixture was in the range of 2-3. Once applied to the skin, the pH of the mixture would still remain low, roughly at a range of 2.2-4. At this pH, the mixture would result in a stinging sensation once applied to the skin. However, such stinging sensation would be reduced or eliminated by the presence of lidocaine, which acts to numb the skin as well as to stabilize the low pH (in view of the buffering capacity of skin tissue at pH 5-5.5, which tends to raise the pH).

For the investigation work, two levels of pepsin were employed: 210 IU/7g gel and 420 IU/7g gel which equates to 30 IU/ml of gel and 60 IU/ml of gel. The Clinical Data resulting from testing of these formulations is provided below in Example 4.

### Example 4

### Clinical Studies: Debridement With Pepsin Instant Activated Gel

The “instant activated” debriding gel of Example 3 was tested on necrotic porcine skin tissue. The purpose of this study was two-fold:

1) To determine the debriding efficacy on dead pig skin of using the two experimental pepsin enzyme gel preparations, 30 IU/ml and 60 IU/ml potency, following application of the pre-debriding ointment of Example 1 and the use of the method of the invention (i.e. the heating step) for 4 hours.

2) To determine the debriding efficacy on dead pig skin of using the two pepsin enzyme gel preparations (30 IU/ml gel and 60 IU/ml gel) without pre-treatment with the pre-debriding ointment or method.

All samples of this study were run in duplicate, identified as samples “a” and “b”. The samples that were pre-treated with the pre-debriding ointment (as described above) were subjected to such pre-treatment for 4 hours at 114-119° F. (45.6-48.3° C.). Debriding status was checked every 12 hours and photos were taken after washing the skin samples in tap water. The enzyme gel was reapplied each time and the samples were incubated at 100±4° F. (37.8±2.2° C.). The 12 hour cycles were continued until all the samples showed full debridement (score of 10). The experiment was ended at 60 hours of debriding (5 cycles).

The results of this study are summarized in Table 4. Samples 1 were pre-treated with the pre-debriding composition while samples 2 were not. The table below shows the debriding scores (a total of epidermal and dermal) on a scale of 0-10.

TABLE 4

| | Debriding Score | | | | | |
|---|---|---|---|---|---|---|
| | 12 | 24 | 36 | 48 | 60 | |
| Sample | Hours | Hours | Hours | Hours | Hours | Total |
| Control a | 0 | 0 | 0 | 0 | 0.5 | |
| Control b | 0 | 0 | 0 | 0.5 | 0.5 | |
| Average Control | | | | | 0.5 | 5% |
| Pepsin 30 IU 1a | 0 | 0 | 0 | 0 | 3 | |
| Pepsin 30 IU 1b | 0 | 0 | 1 | 1 | 2 | |
| Average pepsin | | | | | 2.5 | 25% |
| 30 IU, without | | | | | | |
| pre-debriding | | | | | | |
| Pepsin 30 IU 2a | 1 | 1 | 4 | 5 | 8 | |
| Pepsin 30 IU 2b | 0 | 0.5 | 2 | 3.5 | 9 | |
| Average pepsin | | | | | 8.5 | 85% |
| 30 IU, with | | | | | | |
| pre-debriding | | | | | | |
| Pepsin 60 IU 1a | 0.5 | 2 | 3.5 | 4 | 6 | |
| Pepsin 60 IU 1b | 0.5 | 3 | 5 | 6.5 | 7 | |
| Average pepsin | | | | | 6.5 | 65% |
| 60 IU, without | | | | | | |
| pre-debriding | | | | | | |
| Pepsin 60 IU 2a | 0.5 | 5 | 8 | 9 | 10 | |
| Pepsin 60 IU 2b | 0.5 | 6 | 8.5 | 9 | 10 | |
| Average pepsin | | | | | 10 | 100% |
| 60 IU, with | | | | | | |
| pre-debriding | | | | | | |

### CONCLUSION /

The controls showed little change, as expected in the 60 hour time frame. Samples in which 30 IU/m1 pepsin gel was used showed fair debriding (25%) alone, but a very good (85%) debriding when preceded with treatment using the pre-debriding ointment/warming step. The 60 IU/m1 pepsin gel showed good debriding alone (65%), but excellent debriding (100%) with the pre-debriding treatment.

### Example 5

### Clinical Studies With Papain/Urea Debridement Composition

A clinical study was conducted to examine the efficacy of the pre-debridement composition of the invention with a debridement composition comprising a papain/urea formulation commercially available under the name Kovia™ (Stratus Pharmaceuticals). The details of the samples used in the trial are summarized below in Table 5.

TABLE 5

| | Treated | |
|---|---|---|
| | with pre- | |
| | debriding | |
| Sample | ointment | Exposure time/temperature |
| 1 | NO | Wound Temp (30-34° C.)/6.5 hr. |
| 2 | NO | Wound Temp (30-34° C.)/6.5 hr. |
| 3 | NO | Warmer device Temp (46-49° C.) 4 hr + 2.5 hr |
| | | at 30-34° C. |
| 4 | NO | Denaturation Temp (72° C./45 min) + 5.75 hr |
| | | at 30-34° C. |
| 5 | YES | 6.5 hrs at Wound Temp (30-34° C.) |
| 6 | YES | 6.5 hrs at Wound Temp (30-34° C.) |
| 7 | YES | 6.5 hrs at Wound Temp (30-34° C.) |
| 8 | YES | 6.5 hrs at Wound Temp (30-34° C.) |
| 9 | YES | 2 hr at warmer device temp (46-49° C.) + 4.5 hr |
| | | at 30-34° C. |
| 10 | YES | 2 hr at warmer device temp (46-49° C.) + 4.5 hr |
| | | at 30-34° C. |
| 11 | YES | 4 hr at warmer device temp (46-49° C.) + 2.5 hr |
| | | at 30-34° C. |
| 12 | YES | 6 hr at warmer device temp (46-49° C.) + 0.5 hr |
| | | at 30-34° C. |

Where a pre-debriding ointment was used, the application was maintained for 6.5 hours. For this study, the porcine skin specimens were sandwiched in Telfa™ dressing and dampened with 5 ml water to maintain 100% humidity in a sealed (Ziploc™) bag. The pre-debriding ointment was visible on samples 5-8 at end of 6.5 hrs. Ointment was not visible on samples 9-12 as it largely penetrated into the skin.

After the pre-debriding treatment period, all samples were treated alike as follows: Apply papain/urea debriding ointment and incubate in damp Telfa™ in Ziploc™ bag. Incubate at wound temperature (30-34° C.) for the time specified. Remove papain/urea and firmly scrape skin to remove detached, loosened tissue. Photograph each sample. Evaluate debriding progress with point system for epidermis and dermis on a scale of 0-20. At a score of 20, debriding is complete, i.e. epidermis is gone and dermis is gelatinized, fragmented, partly liquefied. Reapply papain/urea ointment (Kovia™ Ointment, Stratus Pharmaceuticals, lot#5843, exp. 9/09) to start each debriding cycle. Insert into Telfa™ dressing sandwich and Ziploc™ bag. Begin next incubation.

Debriding scoring was calculated as follows:

Epidermis: 0=Intact and 10=gone (i.e. fully debrided). Each score point represents 10% of the epidermis debrided (i.e. a score of 5 means 50% of the epidermis is gone).

Dermis: 0: unchanged from original dermis. 1 to 5: degrees of softened dermis, opaque. 6 to 7: very softened, more translucent. 8 to 9: gelatinized, clearer, hair appears “longer” as dermis is digested. 10: gelatinized, fragmented, semi-liquid.

The pig skin samples used for this trial were cut from just killed pig and not exposed to scalding water, as is the practice to remove the hair by heat denaturing the skin. The skin was full thickness as it peels off the underlying musculature and includes the fascia. Hair was shaved even with the epidermis. Scores reflect epidermis and dermis debriding only. Underlying fascia acts as a substrate base.

The papain debriding scores at the end of each incubation cycle are summarized in Table 6. The scores are calculated as [epidermis score]+[dermis score]=[total score].

TABLE 6

| Sample | 5 hrs | 17 hrs | 29 hrs | 41 hrs | 53 hrs | 65 hrs | 77 hrs | 100 hrs |
|---|---|---|---|---|---|---|---|---|
| 1 | 0 | 5 + 2 = 7 | 8 + 3 = 11 | 9.5 + 4 = 13.5 | 10 + 4 = 14 | 10 + 4 = 14 | 10 + 5 = 15 | 10 + 5.5 = 15.5 |
| 2 | 0 | 4 + 1 = 5 | 6 + 3 = 9 | 8.5 + 4 = 12.5 | 9.5 + 4.5 = 14 | 9.8 + 4.5 = 14.3 | 10 + 5 = 15 | 10 + 5.5 = 15.5 |
| 3 | 0 | 1.5 + 0 = 1.5 | 8 + 3 = 11 | 8 + 3.5 = 11.5 | 8.5 + 4 = 12.5 | 9 + 5 = 14 | 9 + 5.5 = 14.5 | 9 + 6 = 15 |
| 4 | 0 | 0.5 + 0 = 0.5 | 2 + 2 = 4 | 2 + 3 = 5 | 4 + 4 = 8 | 5 + 5 = 10 | 5 + 6.5 = 11.5 | 5.5 + 9 = 14 |
| 5 | 0 | 1.5 + 1 = 2.5 | 6 + 3 = 9 | 8.5 + 4 = 12.5 | 9 + 5 = 14 | 9.5 + 4.5 = 14 | 9.8 + 5.2 = 15 | 10 + 6 = 16 |
| 6 | 0 | 0.5 + 0 = 0.5 | 4 + 3 = 7 | 9 + 3 = 13 | 9.5 + 4.5 = 14 | 10 + 5 = 15 | 10 + 5 = 15 | 10 + 6 = 16 |
| 7 | 0 | 0.5 + 0 = 0.5 | 6 + 3 = 9 | 9.5 + 4 = 13.5 | 10 + 4 = 14 | 10 + 4.5 = 14.5 | 10 + 5.5 = 15.5 | 10 + 7 = 17 |
| 8 | 0 | 0.5 + 0 = 0.5 | 5 + 3 = 8 | 9.5 + 4 = 13.5 | 10 + 4 = 14 | 10 + 4.5 = 14.5 | 10.5.5 = 15.5 | 10 + 7 = 17 |
| 9 | 1 | 7 + 3 = 10 | 8 + 5 = 13 | 10 + 7 = 17 | 10 + 4 = 18 | 10 + 8.5 = 18.5 | 10 + 9 = 19 | 10 + 10 = 20 |
| 10 | 1 | 6 + 3 = 9 | 6 + 6 = 12 | 9.5 + 7 = 16.5 | 10 + 8 = 18 | 10 + 8.5 = 18.5 | 10 + 9 = 19 | 10 + 10 = 20 |
| 11 | 1.5 | 7 + 5 = 12 | 8 + 7 = 13 | 10 + 8 = 18 | 10 + 9 = 19 | 10 + 9.5 = 19.5 | 10 + 10 = 20 | 10 + 10 = 20 |
| 12 | 0.2 | 4 + 2 = 6 | 9 + 7 = 16 | 10 + 7.5 = 17.5 | 10 + 8.5 = 18.5 | 10 + 8.5 = 18.5 | 10 + 9.5 = 19.5 | 10 + 10 = 20 |

Observations

1. The results of samples 1 and 2, which are duplicates, and samples 5-8, which are quadruplicates, are closely similar within each group, within 1 score point starting at 41 hrs after a little variability early on.

2. The average scores of samples 1 and 2 and the score of sample 3 are similar, showing that the temperature difference between wound temp (30-34° C.) and the “warmer device” temp (46-49° C.), in the absence of pre-debriding ointment, does not change the subsequent debriding efficacy. The low scores of sample 4 may be the result of high heat desiccating and hardening the tissue.

3. The two averages of samples 1,2 and 5,6,7,8 at each time point are very similar, showing that at wound temp (30-34° C.) the pre-debriding ointment does not affect the subsequent debriding.

Comparing against matched times of samples 1 and 2 (wound temp (30-34° C.) and no pre-debriding ointment), the following figures are calculated:

4. The results of 2 hrs exposure to both the pre-debriding ointment and the “warmer device” temperature (46-49° C.) clearly show improved subsequent debriding of 14% (at 29 hrs), 29% (at 41 hrs), 29% (at 53 hrs), 31% (at 65 hrs), 27% (at 77 hrs) and 29% (at 100 hrs). Overall average between 41-100 hours: 29%.

5. The results of 4 hrs exposure to both the pre-debriding ointment and the “warmer device” temperature (46-49° C.) clearly show markedly improved subsequent debriding of 30% (at 29 hrs), 38.5% (at 41 hrs), 36% (at 53 hrs), 38%(at 65 hrs), 33% (at 77 hrs) and 33% (at 100 hrs). Overall average between 41-100 hours: 35.7%

6. The results of 6 hrs exposure to both the pre-debriding ointment and the “warmer device” temperature (46-49° C.) clearly show markedly improved subsequent debriding of 60% (at 29 hrs), 35% (at 41 hrs), 32% (at 53 hrs), 31% (at 65 hrs), 30% (at 77 hrs) and 29% (at 100 hrs). Overall average between 41-100 hours: 31.4%.

### CONCLUSIONS

From the above data, the following conclusions can be drawn:

1. Subjecting non-viable skin to “warmer device” temperature (46-49° C.) up to 4 hours does not affect subsequent debriding, compared to normal wound temp (30-34° C.).

2. Subjecting non-viable skin to the pre-debriding ointment at normal wound temp (30-34° C.) does not affect subsequent debriding, compared to normal wound temp (30-34° C.) and without treatment with the pre-debriding ointment.

3. Subjecting non-viable skin to Pre-Debriding ointment AND “warmer device” temperature (46-49° C.) for 2 hours does improve the speed and effectiveness of debriding ointment subsequently applied by 29%.

4. Subjecting non-viable skin to the pre-debriding ointment and the “warmer device” temperature (46-49° C.) for 4 hours does improve the speed and effectiveness of debriding ointment subsequently applied by 35.7%.

5. Subjecting non-viable skin to pre-debriding ointment and “warmer device” temperature (46-49° C.) for 6 hours does improve the speed and effectiveness of debriding ointment subsequently applied by 31.4%.

The similarity of the results at time periods 2, 4 and 6 hrs indicate that treatment with the pre-debriding ointment and warmer device (46-49° C.) can be as short as 2 hours, but optimally about 4 hours. Attention should be paid to patient comfort and safety (including lidocaine anesthesia period, nursing convenience, as well as optimal results).

Further Testing

The testing of samples 1,2,3,5,6 controls was continued to see how long it took to achieve complete debriding, after the samples with the pre-debriding ointment and the “warmer device” finished the debriding process. To be ahead of the controls from 29-35.7% is one thing; finding out just how much longer it takes to finish debriding the controls is another issue of very practical importance. These samples have pepsin or papain/urea ointment re-applied every 12 hours after inspection and photos taken. This was continued until all samples were debrided to a score of 20. Samples were held at 30-34° C. in a Telfa™ “sandwich” inside the Ziploc™ bag with 5 ml water to maintain 100% humidity.

The results of this further testing are presented in Table 7, using the same scoring convention as above.

TABLE 7

| Sample | 112 hrs | | 124 hrs | | 136 hrs | | 148 hrs | 160 hrs | | 172 hrs | | 184 hrs |
|---|---|---|---|---|---|---|---|---|---|---|---|---|
| 1 | 10 + 6.5 = | 16.5 | 10 + 8 = | 18.5 | 10 + 8.5 = | 18.5 | 10 + 9 = 10 | 10 + 9.5 = | 19.5 | 10 + 10 = | 20 | — |
| 2 | 10 + 6.5 = | 16.5 | 10 + 8 = | 18.5 | 10 + 8.5 = | 18.5 | 10 + 9 = 19 | 10 + 9.5 = | 19.5 | 10 + 10 = | 20 | — |
| 3 | 9.5 + 6.5 = | 16 | 10 + 7 = | 17 | 10 + 8 = | 18 | 10 + 8.5 = 18.5 | 10 + 9.5 = | 19.5 | 10 + 10 = | 20 | — |
| 5 | 10 + 7 = | 17 | 10 + 7 = | 17 | 10 + 8 = | 18 | 10 + 8.5 = 18.5 | 10 + 9 = | 19 | 10 + 9.5 = | 19.5 | 10 + 10 = 20 |
| 6 | 10 + 7 = | 17 | 10 + 7 = | 17 | 10 + 7.5 = | 17.5 | 10 + 8.5 = 18.5 | 10 + 9 = | 1 | 10 + 9.5 = | 19.5 | 10 + 10 = 20 |

Observations

The additional treatment time for the controls compared to the test samples (100 hours):

Controls #1 and #2 (duplicates—no pre-debriding ointment, wound temp, 30-34° C.): Required 72 hours additional time.

Control #3 (no pre-debriding ointment, “warmer device” temperature, 46-49° C. for 4 hours): Required 72 hours additional time.

Controls# 5 and 6 (pre-debriding ointment, wound temp 30-34° C.): Required 84 hours additional time.

Conclusions of Papain Debridinq With and Without Pre-Debridinq

Based on debriding completion time vs. pre-debriding ointment treated samples, controls 1, 2 and 3 took 72% more time (under the present treatment situation). Controls 5, 6 took 84% more time.

In other words, “72% more time” means that use of the pre-debriding ointment in conjunction with the “warming device” reduced debriding time by 42%. Similarly, “84% more time” means that use of the pre-debriding ointment and the “warming device” reduced debriding time by 45.6%.

Although the invention has been described with reference to certain specific embodiments, various modifications thereof will be apparent to those skilled in the art without departing from the purpose and scope of the invention as outlined in the claims appended hereto. Any examples provided herein are included solely for the purpose of illustrating the invention and are not intended to limit the invention in any way. Any drawings provided herein are solely for the purpose of illustrating various aspects of the invention and are not intended to be drawn to scale or to limit the invention in any way. The disclosures of all prior art recited herein are incorporated herein by reference in their entirety.

### REFERENCES

1) Usha, R. and Ramasami, T., The Effects of Urea and n-Propanol on Collagen Denaturation: Using DSC, Circular Dicroism and Viscosity, Thermochemica Acta, 409, 201-206, (2004).

2) Leikina, E., Mertts, V., Kuznetsova, and Leikin, S., Type I Collagen is Thermally Unstable at Body Temperature, Biophysics, 10.1073/pnas.032307099, (2002).

3) Miles, C. A., and Ghelashvili, M., Polymer-in-a-Box Mechanism for the Thermal Stabilization of Collagen Molecules in Fibers, Biophysics J., June, pp. 3243-3252, vol 76, No. 6, (1999).

4) Gross, Jerome, Thermal Denaturation of Collagen in the Dispersed and Solid State, Science, 28, Vol. 143, no. 3609, pp 960-961, (1964).

5) Matsushita, S; Deki, S; Mizuhata, M; Sugita, M; Kanaji, Y, Temperature Dependence of pH for Solubilized Collagen Solutions, J. Applied Polymer Science vol. 54, no. 10, pp. 1561-1566, (1994).

6) Pelle, M. T.; Miller, 0.F., Debridement of Necrotic Eschar with 40% Urea Paste Speeds Healing of Residual Limbs and Avoids Further Surgery, Arch Dermatology, Vol 137, (2001).

7) Kling, J. W. and Riggs, L. A., Editors, Woodworth & Schlosberg's Experimental Psychology, 3rd. edition, pp. 154-166, (1971), Holt, Rinehart and Winston, Inc., New York, N.Y.

8) Anchordoguy, T. J. et al., Effects of Protein Perturbants on Phospholipid Bilayers, Arch. Biochem. Biophys. 283: 356 (1990).

9) Behl, C. R. et al., Age and Anatomical Site Influence on Alkanol Permeation of Skin of the Male Hairless Mouse, J. Soc. Cosmet. Chem., 35: 237 (1984).

10) Rowe, E. S., Lipid Chain Length and Temperature Dependence of Ethanol-Phosphatidyl Choline Interactions, Biochemistry 22: 3299 (1983).

11) Wohlrab, Chemical Abstracts, AN 1993:160285.

12) Mahjour et al., Chemical Abstracts, AN 1989:13572.

13) Gaspulin et al., Chemical Abstracts, AN 1993:546514.

We claim:

1. A pre-debriding composition comprising:
a first chaotropic component, wherein the first chaotropic component is: urea; N-alkyl-substituted urea or N,N-dialkyl-substituted urea having a C₆ to C₉ alkyl group; amides of formula (R²)₂N—CO—R¹—R³, wherein R¹ represents a chemical bond or a C₁ to C₁₄ alkyl group, and R² and R³ are independently hydrogen or CH₃; dimethyl sulfoxide (DMSO); or a combination thereof;
at least one of: an analgesic component, an exfoliating component, an unsaturated fatty acid, and a collagen re-naturing inhibitor; and,
one or more of pharmaceutically acceptable thickening agents, emulsifiers, preservatives, carriers, diluents, adjuvants and excipients.

2. The pre-debriding composition of claim 2, wherein the first chaotropic component is present in a concentration of about 15 to 40 wt %.

3. The pre-debriding composition of claim 1, further comprising a second chaotropic component and wherein the second chaotropic component comprises an alcohol selected from C₂ to C₈ aliphatic alcohols, aromatic alcohols, and combinations thereof and wherein the alcohol is present in the composition in a concentration of about 3 to 5 wt %.

4. The pre-debriding composition of claim 1, wherein the analgesic component comprises at least one of benzocaine, butamben picrate, dibucaine, dimethisoquin, diclonyne, lidocaine, pramoxine, tetracine, and pharmaceutically acceptable salts thereof, and combinations thereof and wherein the analgesic component is present in a concentration of about 0.2 to 20 wt %.

5. The pre-debriding composition of claim 1, wherein the unsaturated fatty acid component is at least one of oleic acid, linoleic acid, linolenic acid, lactic acid, malic acid, mandelic acid, or a combination thereof, and wherein the unsaturated fatty acid is present in a concentration of about 1 to 5 wt %.

6. The pre-debriding composition of claim 1, wherein the collagen re-naturing inhibitor comprises one or more of glucose, related glucose sub-unit products, protein solubilization and emulsification agents, and combinations thereof.

7. The pre-debriding composition of claim 1, wherein the collagen re-naturing inhibitor is glucose, n-propanol, sodium lauryl sulfate or a combination thereof.

8. The pre-debriding composition of claim 7, wherein: glucose is present in a concentration of about 1-5 wt %; n-propanol is present in a concentration of about 2-10 wt %; and, sodium lauryl sulfate is present in a concentration of about 0.005-0.015%.

9. A method of treating a wound site prior to debridement, the method comprising:
a) applying to the wound site, at least one first chaotropic agent and at least one analgesic agent, for reducing the collagen denaturation temperature at the wound site, wherein the at least one first chaotropic agent is: urea; N-alkyl- substituted urea or N,N-dialkyl-substituted urea having a C₆ to C₉ alkyl group; amides of formula (R²)₂N—CO—R¹—R³, wherein R¹ represents a chemical bond or a C₁ to C₁₄ alkyl group, and R² and R³ are independently hydrogen or CH₃; dimethyl sulfoxide (DMSO); or combinations thereof; and,
b) applying heat to the wound site following application of the pre-debriding composition, said heat being applied for a period of time to cause denaturation of collagen at the wound site.

10. The method of claim 9, wherein step (a) further comprises applying to the wound site a second chaotropic agent, wherein the second chaotropic agent is an alcohol selected from C₂ to C₈ aliphatic alcohols, aromatic alcohols, and combinations thereof.

11. The method of claim 10, wherein the first chaotropic agent is urea and the second chaotropic agent is n-propanol.

12. The method of claim 9, wherein the analgesic agent comprises at least one of benzocaine, butamben picrate, dibucaine, dimethisoquin, diclonyne, lidocaine, pramoxine, tetracine, and pharmaceutically acceptable salts thereof, and combinations thereof.

13. The method of claim 10, wherein step (a) further comprises applying to the wound site at least one unsaturated fatty acid, at least one exfoliating agent, and at least one collagen re-naturing inhibitor.

14. The method of claim 9, wherein step (a) comprises applying a composition in the form of an ointment or a gel to the wound site.

15. The method of claim 9, wherein step (b) comprises heating the wound site to a temperature of about 10° C. above body temperature.

16. The method of claim 15, wherein step (b) comprises increasing the temperature of the wound site gradually over a period of 2-6 hours.

17. The method of claim 16, wherein, in step (b), the rate of temperature increase in the first 30 to 60 minutes is less than the rate of increase after said 30 to 60 minute period.

18. A system for heating a wound site following application of a pre-debriding composition to cause denaturation of collagen at the wound site, the system comprising:
i) a first dressing layer for placement over the wound site, the first dressing layer comprising a non-absorbent material;
ii) a temperature measuring or sensing means for positioning over the first dressing layer;
iii) a second dressing layer, for placement over the temperature measuring or sensing means and over the first dressing layer, the second dressing layer comprising an absorbent material having a non-adherent layer, the second dressing layer including a fenestration corresponding to the wound site being treated; and,
iv) a heating means for heating the wound site.

19. A kit for providing a pre-debriding treatment at a wound site comprising:
a) a pre-debriding composition according to claim 1;
b) a first dressing layer comprising a non-absorbent material;
c) a temperature measuring or sensing means;
d) a second dressing layer comprising an absorbent material having a non-adherent layer; and,
e) a heating means for heating the wound site.

20. The kit according to claim 19, further comprising a debriding composition.

21. The kit according to claim 20, wherein the debriding composition is provided in two parts comprising a gel part and an active substance containing powder part, and wherein the debriding composition is provided in a single package having first and second compartments separated by a breakable divider, the first compartment containing the gel part of the debriding composition and the second compartment containing the active substance containing powder part of the debriding composition.

22. The kit according to claim 21, wherein the active substance containing powder part comprises a powdered form of an enzyme component and a powdered form of an analgesic agent and wherein the enzyme component comprises one or more proteolytic enzymes in their zymogen form.

23. The kit according to claim 22, wherein the one or more enzymes are pepsin, fruit proteolytic enzymes or bacterially derived proteolytic enzymes or a combination thereof.

24. The kit according to claim 23, wherein the fruit proteolytic enzymes are one or more of papain, actinidin, bromelin, or ficin.

25. The kit according to claim 24, wherein the enzyme component further comprises fructose and/or glucose and at least one antimicrobial agent.

26. The kit according to claim 25, wherein the at least one antimicrobial agent is methylglyoxal or 1,3-dihydroxyacetone.

27. A composition for enzymatic debriding, wherein the composition is provided in two components consisting of a gel composition and a powder composition, wherein the powder composition comprises one or more proteolytic enzymes in their zymogen form and wherein the gel composition is adapted to activate one or more zymogens when the two components are mixed.

28. The composition according to claim 27, wherein the one or more enzymes are pepsin, fruit proteolytic enzymes or bacterially derived proteolytic enzymes or a combination thereof.

29. The composition according to claim 28, wherein the fruit proteolytic enzymes comprise one or more of papain, actinidin, bromelin or ficin and wherein the composition further comprises comprising fructose and/or glucose and at least one antimicrobial agent.

30. The composition of claim 27, wherein the at least one antimicrobial agent is methylglyoxal or 1,3-dihydroxyacetone.

31. The composition of claim 27, wherein the powder composition further comprises an analgesic agent in powder form.

32. A package for the debriding composition of claim 27, wherein the package comprises two compartments separated by a breakable divider, one of the compartments containing the gel composition and the other of the compartments containing the powder composition.
